(19) United States
(12) Patent Application Publication
(10) Pub. No.: US 2022/0110993 A1
(43) Pub. Date: Apr. 14, 2022

(54) COMPOSITIONS FOR TREATMENT OF JAW PAIN, TEMPOROMANDIBULAR JOINT AND MUSCLE DISORDER AND BRUXISM

(71) Applicant: LAB-O Corp., Boca Raton, FL (US)

(72) Inventors: Layne Lieberman, Boca Raton, FL (US); Alexander Anapol, Philadelphia, PA (US)

(73) Assignee: LAB-O Corp., Boca Raton, FL (US)

(21) Appl. No.: 17/492,147

(22) Filed: Oct. 1, 2021

Related U.S. Application Data
(60) Provisional application No. 63/090,628, filed on Oct. 12, 2020.

(51) Int. Cl. A61K 36/53; A61K 9/06; A61K 47/44; A61K 31/125; A45D 34/04; A61P 29/02

(52) U.S. Cl. CPC A61K 36/53; A61K 9/06; A61P 29/02; A61K 31/125; A45D 34/04; A61K 47/44

(57) ABSTRACT

The present disclosure provides a system for modulating jaw pain and bruxism. The system includes a tube having a composition formulated as a topical balm comprising a botanical formulation and a topically acceptable carrier and a dispensing tube having an applicator tip.

### CLAIM FOR PRIORITY

This application claims priority under 35 U.S.C. § 119(e) to U.S. Patent Application Ser. No. 63/090,628 filed on Oct. 12, 2020, which is hereby incorporated by reference in its entirety.

### TECHNICAL FIELD

This invention relates to products and methods for treating jaw pain, temporomandibular joint and muscle disorder, and related bruxism.

### BACKGROUND OF THE INVENTION

Jaw pain has several etiologies including but not limited to temporomandibular joint disorders, inflammation of underlying muscles, and bruxism (clenching and grinding of teeth). The temporomandibular joint (TMJ) acts like a sliding hinge, connecting a jawbone to the skull. TMJ disorders, a type of temporomandibular disorder, can cause pain in the jaw joint and in the muscles that control jaw movement. Over 40 million Americans have jaw pain related to the TMJ. Many people rely on uncomfortable and costly nightguards, Botox, or major facial surgery for relief. The pain associated with TMJ disorders may be due to a combination of factors, such as genetic predisposition, arthritis, or jaw injury. Some people who have jaw pain also tend to clench or grind their teeth (bruxism), although many people habitually clench or grind their teeth without developing TMJ disorders. Conventional treatments of jaw pain include mouth guards, nonsteroidal anti-inflammatory drugs (NSAIDs), crown and bridge work to balance the bite, orthodontics to change the bite, and even surgery. These treatments tend to be invasive, inconvenient and/or costly. In most cases, the pain and discomfort associated with TMJ disorders is temporary. But it can be chronic as well. Thus, there is a need for self-managed care or nonsurgical treatments for TMJ disorders and associated pain.

### SUMMARY OF THE INVENTION

The present disclosure provides a system for relieving jaw pain or bruxism, comprising a tube having an applicator tip and a composition formulated as a topical balm dispensable from the tube through the applicator tip, the composition comprising a botanical formulation, the botanical formulation comprising an effective amount of menthol, an effective amount of a terpenoid (e.g., camphor), an effective amount of a lavender oil, an effective amount of a wintergreen oil, and an effective amount of a ginger root oil. In some embodiments, the botanical formulation comprises about 1.25 to 16 wt % menthol, 0.1-7 wt % camphor, 2-10 wt % lavender oil, 0-11 wt % wintergreen oil, and 1-8 wt % ginger root oil, based upon the total weight of the composition.

In certain embodiments, the lavender oil is obtained from the leaf, root, flowers, bud or plant of Lavandula angustifolia, Lavandula burnamii, Lavandula dentate, Lavandula dhofarensis, Lavandula latifolia, Lavandula officinalis, Lavandula stoechas, or combinations thereof.

In some embodiments, the composition further comprises a topically acceptable carrier. In certain embodiments, the carrier includes jojoba oil and beeswax. In particular embodiments, the carrier comprises 50-74 wt % jojoba oil. In certain embodiments, the carrier comprises 64-74 wt % jojoba oil and 10-20 wt % beeswax, based upon the total weight of the composition.

In some embodiments, the composition further comprises one or more of willow bark oil, clove oil, lemon oil, cinnamon bark oil, rosemary oil, and eucalyptus oil.

In an aspect, the present disclosure provides a composition formulated as a topical balm for administration on a human subject, comprising a botanical formulation consisting of menthol, a terpenoid, lavender oil, wintergreen oil, and ginger root oil; and a topically acceptable carrier (e.g., a hydrophobic or amphiphilic carrier).

In another aspect, the present disclosure provides a method of alleviating jaw pain or bruxism in a human subject, comprising applying to the jaw area of the subject the topical composition described herein.

Other aspects, embodiments, and features will be apparent from the following description, the drawings, and the claims.

### BRIEF DESCRIPTION OF THE DRAWING

FIG. 1 is a diagram showing a system 100 including a tube 101 having an applicator tip 102 and a composition 103 formulated as a topical balm dispensable from the applicator tip.

### DETAILED DESCRIPTION OF THE INVENTION

The present disclosure provides novel topical botanical compositions and devices for treating jaw pain, TMJ disorders and bruxism, without the adverse side effects typically associated with conventional topical analgesics. Prior natural treatments (such as prior essential oil blends), while providing soothing or moisturizing properties, do not effectively provide pain relief. Conversely, prior topical analgesics may provide pain relief, but they often result in a stinging or burning sensation, eye-watering effects, and dry skin. These analgesics therefore do not lend themselves well to night-time use around the jaw and delicate facial area. The strong medicinal odors emanating from such products also result in poor sleeping patterns and disturbance to partners.

The present compositions contain a unique combination of ingredients derived from plant sources as set forth in more detail below. The compositions can include a botanical formulation and a dermatologically acceptable carrier. In some embodiments, the compositions are purely natural (e.g., botanically derived). In certain embodiments, the compositions are free of synthetic ingredients or additives. The compositions can be regulated by the FDA under an over-the-counter (OTC) monograph. The compositions can be provided as, for example, a balm. As used herein, a balm refers to any viscous topical composition including an ointment, a salve, a cream, or a gel. One example of the claimed compositions is the product TJz Balm™.

The present formulations achieve both the pain relief of a topical analgesic and the pleasant and skin-safe properties of prior natural treatments. The present formulations, with their unique combination of botanical ingredients, are pleasantly scented, soothing, sleep friendly, and moisturizing. They do not cause side effects such as stinging sensation, watery eyes, risk of migration into the eye area, dry skin, and sleep disturbance. Moreover, while conventional topical analgesics are applied using one's hands or fingers, risking ingestion or migration to the eye area (for example by rubbing or touching one's eyes) and requiring cleanup, the present composition is dispensed from an applicator which also can serve as a mechanical massage tool. The applicator minimizes the need for post-use cleanup and reduces the risk of ingestion or inadvertent migration to the eye area.

### I. Botanical Formulations

A botanical formulation herein can include a combination of botanically derived ingredients (e.g., oils) and other products that are specifically selected to provide pain relief, reduce or eliminate bruxism, and improve sleeping patterns via counter-irritant properties, a heating sensation, a cooling sensation, muscle relaxant effects, and aromatherapeutic effects. The botanical formulation can be mixed with a carrier to make the final composition, which can then be massaged into the jaw and surrounding areas via a specially designed applicator tip.

In certain embodiments, the botanical formulation is made of ingredients extracted from organic plants. In certain embodiments, the formulation can include synthetic versions of naturally derived compounds that provide the same effects. In other embodiments, other ingredients may be combined or substituted so long as the resultant composition provides the same or similar effect of relieving jaw pain or bruxism.

The botanical formulation can include effective amounts of menthol, a terpenoid, a lavender extract or oil, a wintergreen extract or oil, and a ginger root extract or oil. Each of these ingredients are further described in detail below.

A. Menthol Crystals

The main form of menthol occurring in nature is (−)-menthol, which is assigned the (1R,2S,5R) configuration as shown below. Menthol is the solid constituent of the oil of mint and gives mint its characteristic smell. Menthol crystals possess a powerful peppermint aroma and give a cooling sensation when applied to the skin as balms or rubs.

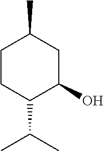

Menthol belongs to the class of organic compounds known as menthane monoterpenoids, which have a structure based on the o-, m-, or p-menthane backbone. P-menthane has a cyclohexane ring with a methyl group and a (2-methyl)-propyl group at the 1 and 4 ring position, respectively. The o- and m-menthanes are rarer, and presumably arise by alkyl migration of p-menthanes. See, e.g., pubchem.ncbi.nlm.nih.gov/compound/Menthol.

Menthol's analgesic properties are mediated through a selective activation of K-opioid receptors. See, e.g., Galeotti et al., (2002), Neurosci Lett. 322 (3):145-8. Menthol also blocks voltage-sensitive sodium channels, reducing neural activity that may stimulate muscles. See, e.g., Haeseler et al., (2002), Eur J Anaesthes. 19 (8):571-9.

Menthol crystals can be sourced from corn mint (Mentha arvensis) in an essential oil extraction. They can also be steam distilled from peppermint oil. These clear and colorless crystals are soluble in both oil and alcohol. Menthol crystals can be used at 2-20% in balms, liniments, and rubs to lend a cooling sensation.

B. Wintergreen Oil

Wintergreen oil is a pale yellow or pinkish fluid with a strong sweet and woody odor. Wintergreen oil is traditionally extracted by fermentation of the natural material from the source plant (e.g., the leaves of wintergreens), followed by distillation to obtain a purified oil product. The primary compound (about 98%) found in the oil is methyl 2-hydroxybenzoate or methyl salicylate. Minor components in the oil include α-pinene, myrcene, δ-3-carene, limonene, 3,7-guaiadiene, and δ-cadinene). See, e.g., Gurung, “Analysis of Wintergreen Oil Extracted from Leaves of Wintergreen in Distilled Units,” 2007.

Methyl salicylate is a methyl ester of salicylic acid with the following structure:

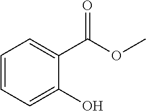

This compound is naturally produced by many species of plants, particularly wintergreens (e.g., Gaultheria procumbens). Methyl salicylate is used as a rubefacient and analgesic in deep heating liniments. See, e.g., pubchem.ncbi.nlm.nih.gov/compound/Wintergreen %20oil.

In the past, research into wintergreen oil or methyl salicylate as a topical pain reliever has shown mixed results. Although some authors have suggested using wintergreen oil as a potential alternative treatment for lower back pain, other authors have noted that larger trials need to be performed to assess efficacy. See, e.g., Hebert et al., J Ahern Compl Med. (2014) 20(4):219-20; Derry et al., Cochrane Database Syst Rev. (2014) 2014(11):CD007403.

C. Lavender Oil

Lavender oil is an essential oil derived from the lavender plant. Lavender oil can be extracted from Lavandula angustifolia (English lavender or common lavender), Lavandula burnamii, Lavandula dentata (French lavender), Lavandula dhofarensis, Lavandula latifolia (Portuguese lavender), Lavandula officinalis, Lavandula stoechas (Spanish lavender), lavandin, other garden varieties of lavender, or combinations thereof. The oil is made by distilling the lavender buds, capturing the steam, and condensing the steam into a liquid. Lavender oil can be taken orally, applied to the skin, and inhaled through aromatherapy. Lavender oil can benefit the skin in numerous ways.

The main chemical components of lavender oil are α-pinene, limonene, 1,8-cineole, cis-ocimene, trans-ocimene, 3-octanone, camphor, linalool, linalyl acetate, caryophyllene, terpinen-4-ol and lavendulyl acetate. The compounds responsible for the pleasant scent of lavender are linalyl acetate and linalool. Linalool (3, 7-Dimethyl-1, 6-octadien-3-ol) refers to two enantiomers of a naturally occurring terpene alcohol found in many flowers and spice plants and has a sedative effect. Linalool is shown below.

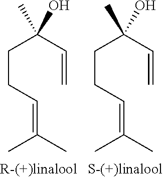

Linalool belongs to the class of organic compounds known as acyclic monoterpenoids. See, e.g., pubchem.ncbi.nlm.nih.gov/compound/linalool. Relevant to its mechanism of action, linalool behaves as a competitive antagonist of glutamate and as a non-competitive antagonist of NMDA receptors in brain cortical membranes. Linalool is believed to be responsible for lavender oil's therapeutic effect in alleviating skin burns without scarring (Gattefosse, “Aromatherapy,” Penguin Random House, 1993). Moreover, the local anesthetic effects of linalool are reported to be equal to those of procaine and menthol. Id.

It was found that when combining lavender oil with gingerols and other ingredients, the resulting compositions were effective in mitigating or canceling the stinging and eye-watering effect of menthol and camphor and minimizing the medicinal scent of these ingredients without reducing their analgesic properties, while also promoting sleep.

D. Ginger Oil

Ginger oil is derived from the root of the herb Zingiber officinale. Ginger oil has a distinct strong aroma that can be described as warm and spicy. Ginger oil can be extracted from the ginger rhizome through a distillation process. Ginger is abundant in active constituents, such as phenolic and terpene compounds. The phenolic compounds in ginger are mainly gingerols, shogaols, and paradols. In fresh ginger, gingerols are the major polyphenols, such as 6-gingerol, 8-gingerol, and 10-gingerol. Gingerols are beta-hydroxy ketones and members of guaiacols. See, e.g., pubchem.ncbi.nlm.nih.gov/compound/gingerol. A structure of 6-gingerol is shown below:

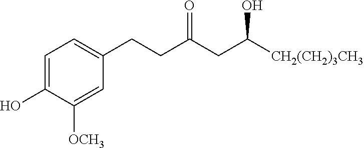

With heat treatment or long-time storage, gingerols can be transformed into corresponding shogaols. A shogaol structure is shown below:

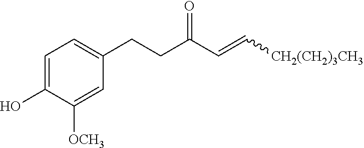

After hydrogenation, shogaols can be transformed into paradols. There are also many other phenolic compounds in ginger, such as quercetin, zingerone, gingerenone-A, and 6-dehydrogingerdione. Moreover, there are several terpene components in ginger, such as β-bisabolene, α-curcumene, zingiberene, α-farnesene, and β-sesquiphellandrene, which are considered to be the main constituents of ginger oil.

It was discovered that when combining ginger oil with lavender oil and other ingredients, the resulting compositions were effective in mitigating or canceling the stinging and eye-watering effect of menthol and camphor and minimizing the medicinal scent of these ingredients without reducing their analgesic properties, while also promoting sleep.

Ginger oil that is topically applied should always be diluted in a carrier oil first such as almond oil, jojoba oil, coconut oil, and avocado oil. The health benefits of ginger oil are the same as that of the herb from which it originates, with the oil being even more beneficial due to its higher gingerol content, a constituent that is mostly reputable for its antioxidant and anti-inflammatory properties. It has a warm, sweet, woody, and spicy scent that has an energizing effect.

E. Camphor Crystals

Camphor is a naturally occurring terpenoid with the chemical formula C10H₁₆O. Specifically, it is a cyclic monoterpene ketone that is bornane bearing an oxo substituent at position 2. Camphor belongs to a group of organic compounds defined as terpenoids, and specifically, terpenoid ketones. It is found in the wood of the camphor laurel (Cinnamomum camphora), a large evergreen tree found in East Asia, also of the unrelated kapur tree (Dryobalanops sp.), a tall timber tree from Southeast Asia. The molecule has two possible enantiomers as shown in the structural diagrams below. The structure on the left is the naturally occurring (+)-camphor ((1R,4R)-bornan-2-one), while its mirror image shown on the right is the (−)-camphor ((1S,4S)-bornan-2-one).

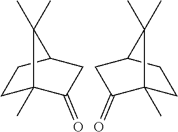

Camphor can appear as a colorless or white crystalline powder with a strong mothball-like odor and has about the same density as water. Camphor oil appears as a colorless liquid with a characteristic odor and has a flash point 125° F. It is insoluble in water and generally less dense than water. Its vapors are heavier than air. D-camphor appears as colorless or white crystals. It sublimes, and has a flash point of 149° F. It burns readily with a bright, smoky flame. It has a penetrating aromatic odor, a pungent, aromatic taste followed by a sensation of cold. Camphor is rapidly absorbed from the mucous membranes and the gastrointestinal tract. It is also absorbed through inhalation, through dermal application, and by nasal instillation. See, e.g., pubchem.ncbi.nlm.nih.gov/compound/Camphor.

Camphor crystals can be obtained from a natural cold extraction process of the camphor tree. The clippings, roots and wood chips of the tree can be processed to produce camphor crystals and camphor oil. Camphor extracted from the wood of the camphor tree is extracted by steam distillation, which yields transparent crystals. It is isolated by passing steam through the pulverized wood and condensing the vapors. Camphor crystallizes from the oily portion of the distillate and is purified by pressing and sublimation. Extraction from the leaves can also produce camphor crystals.

F. Preservative

As an option, a preservative can be added to extend the shelf life of the present formulations and compositions, reduce spoilage, and retain texture, color, or smell of the products. The preservative can be a natural preservative such as vitamin E, rosemary oil, oregano extract, hops, salt, sugar, vinegar, alcohol, diatomaceous earth, castor oil, or wheat germ oil. In certain embodiments, the preservative can also include a synthetic or artificial substance.

### II. Carrier Material

A carrier material can be a single material, a blend of materials selected based on chemical and physical compatibility with the active ingredients or the botanical formulation, the desired viscosity of the final composition, and the ability of the carrier material to give stability to the final composition. The carrier material must have a texture and viscosity that is easily dispensed from the applicator, have a texture that is smooth, not overly greasy, and have a long-lasting effect. The carrier can be a hydrophobic material such as a wax or an oil. Some examples of carrier oils include almond oil, jojoba oil, coconut oil, and avocado oil.

A. Jojoba Oil

Simmondsia Chinensis (Jojoba) seed oil has an ability to mimic the body's natural oils. Accordingly, it does not cause the skin to produce more sebum. Jojoba oil balances skin oil production, making it ideal for all skin types, including oily complexions. The jojoba plant is a hardy, perennial plant that grows in North America. It thrives in harsh, arid climates. The nut of the jojoba plant can be made into an oil. Jojoba oil is gentle enough to be used as a carrier oil for the essential oils in the botanical formulation. Jojoba oil imparts moisturizing properties and has been applied as a remedy for acne, dry skin, and other skin conditions. Jojoba oil contains antimicrobial and antifungal properties. It contains natural forms of vitamin E. Accordingly, it can fight oxidative stress caused exposure to pollutants and other toxins. It is non-comedogenic and hypoallergenic. Jojoba oil is waxy and creates a soothing seal on the skin surface.

B. Beeswax

Beeswax is a natural product made from the honeycomb of the honeybee and other bees. It is hard and breakable when cold but soft and pliable when heated. The mixing of pollen oils into honeycomb wax turns the white wax into a yellow or brown color. Beeswax is used for treating high cholesterol, pain, fungal skin infections, and other conditions. But there is no good scientific research yet to support these uses. In foods and beverages, white beeswax and beeswax absolute (yellow beeswax treated with alcohol) are used as stiffening agents. In manufacturing, yellow and white beeswax are used as thickeners, emulsifiers, and as stiffening agents in cosmetics. Beeswax absolute is used as a fragrance in soaps and perfumes. White beeswax and beeswax absolute are also used to polish pills. Beeswax hydrates, conditions, soothes, and calms the skin. It exfoliates, repairs damage, promotes the skin's regeneration, diminishes the appearance of the signs of aging, soothes itchiness and irritation, and creates a hydrating, long-lasting protective barrier against environmental pollutants and can be useful in treating topical allergies or skin ailments, such as eczema and rosacea.

### III. Methods of Manufacturing

The composition to be dispensed can be prepared by heating a carrier material such as jojoba oil and beeswax in one container. In a separate container, a botanical formulation as described above can be blended, heated and agitated. The carrier material and botanical formulation can then be combined and subsequently cooled to room temperature. The resulting composition can be filled into a container or tube.

Formulations as noted in the examples below are made to comply with the FDA Tentative Final Monograph for OTC topical analgesic-drugs (the “Monograph”), which are required to be safe and effective and have a pleasing scent. All percentages provided herein are in % weight over weight based on the total weight of the composition. Menthol generally is provided at about 2.4% by weight to be effective. Camphor is generally provided at about 1.4% by weight to effectively provide an analgesic effect. Ginger oil is generally provided at about 1-8% by weight, and preferably, 2% by weight for a heating sensation. Lavender is generally provided at about 2-10 wt % to effectively mitigate stinging or burning sensation of the analgesic, and wintergreen is generally provided at about 0-11 wt % for pain relief and to balance the heat and cooling effects of other ingredients. In certain embodiments, menthol is provided as an active ingredient as defined by the Monograph, with jojoba oil, beeswax, wintergreen oil, lavender oil, ginger oil, and camphor as inactive ingredients.

The beeswax and jojoba oil were formulated to provide an effective and safe composition that works effectively to create a consistency that is conducive for being dispensed from the appropriate tube applicator. The resultant product is stable and spreadable without the need for synthetic additives. The composition can be administered to human adults and children using the applicator tip, massaged into the affected area. The composition can be administered before bedtime. For children 12 years of age or younger, a physician can first be consulted before use.

### IV. Applicator Tube and Tip

The design of the applicator tube is important for safety and efficacy of the product and its method of use of the claimed system and method. The composition can sting if it gets in contact with the eyes, and therefore it is desirable to avoid direct contact with hands or fingers that could inadvertently contact one's eye area. The applicator tip is structured as a massage tool to be used on the facial area, for example, when or after the claimed composition is applied, and is important for the effective use of the product. Accordingly, the applicator is structured to have a tube configured to hold the composition, the tube having a tube neck or nozzle, and a tip connected to the nozzle and configured to dispense an effective amount of the composition and accommodate such an effective amount such that it can be applied to a facial area directly from the tip. The tip must also be constructed so that it does not bend or flex when used as a facial area massage tool, and the nozzle or neck between the tube and the tip does not break when using it as a massage tool.

The most common shapes for the tube are cylindrical, but any suitable shape can be used including prismatic, oval, convex, round and square shapes. A squeezable tube can be made of multiple layers of flat sheets of laminated plastic and/or aluminum for example. The laminate can be printed then rolled and sealed on the side to form a tube. The cylinder is then joined to a nozzle or tube neck. In certain embodiments, a tube can be a rigid tube. A tube can be configured to dispense a predetermined amount of the composition. In certain embodiments, the tube can be configured to dispense a flexible amount of composition based on the pressure exerted by the user.

The applicator tube can be made of any suitable material to hold and dispense the claimed composition. The applicator tube can be a portable size that is handheld and easy to store and use. For example, the tube can be made of plastic (e.g. polyethylene, or polypropylene), acrylic, nylon, metal (e.g. aluminum), nylon and or combinations of these materials. In certain embodiments, a tube can be a multi-layer tube such as a 3-layer or 5-layer tube. The tube can be made of plastic, a polyolefin or modified polyolefin. The tube material can include LDPE, ADMER, EVOH, PET or EVAL or a combination of these. The tube can be made of laminate or co-extrusion plastic. The tube can be designed to block air and be well-suited for oil-based formulas with active ingredients. In certain embodiments, a sustainable material can also be used. For example, eco-friendly polyethylene tubes made from sugar cane bio-resin can be used. In other examples, recycled material such as recycled plastic can be used as a tube material. The tube can have a neck or nozzle that is dimensioned to have a smaller diameter than the tube to constrict flow and prevents over-dispensing of the composition within the tube. It can also be configured to receive and attach an applicator tip to the tube.

In certain embodiments, the applicator can be an airless type cosmetic container that is designed to be used to discharge a composition in the cosmetic container according to the pressing and releasing of a button. Specifically, by the continuous operation of a piston and an axial valve during the cosmetic discharge, the opening and closing nozzle can be controlled. Such a design can ensure accuracy and is convenient for use and consistent amounts of a composition can be dispensed as and the outlet of the nozzle is automatically closed after use to prevent leakage, thereby ensuring safety of portable storage and preventing contamination and deterioration of the composition. Such a design is shown for example, in international published application WO2010047486A2.

In certain embodiments, the tube can be multi-layered tube with high barrier properties such as aluminum, poly-foil laminate tube or plastic laminate tubes including an EVOH barrier, or any suitable material which protects against oxygen, chemical solvents, and essential oils for example, that keep product from UV, moisture, and oxygen exposure. These tubes also known as cosmetic tubes, provide protection for a contained composition and increase the composition's shelf life. Sensitive formulas such as those that contain essential oils, SPF, natural cosmetics and skincare formulations are preferably packaged in cosmetic tubes with such properties since these formulations can cause chemical reactions when packaged in a traditional tube. Unlike aluminum tubes, plastic laminate or poly-foil laminate tubes will bounce back or regain their original shape without creasing after being squeezed.

In other embodiments, the applicator can use a squeezable tube. The tube can deform upon squeezing but retain its shape after it is squeezed. In other examples, the tube can deform and remain in a deformed configuration after it is squeezed. Depending on the desired amount, the composition can be dispensed to provide an effective amount to treat a facial area based on the severity of the pain and the size of the area of application.

The applicator tip can be rounded or oval and smooth so that the composition can be massaged into the facial area, e.g. in a smooth or circular motion. The applicator tip can also be made of any suitable material to dispense the claimed composition and apply it directly to a facial area. For example, the tip can be made of plastic (e.g. polyethylene, polypropylene), acrylic, metal (e.g. aluminum), or combinations of these materials.

In certain embodiments, a sustainable material can also be used. For example, eco-friendly polyethylene tubes made from sugar cane bio-resin can be used. In other examples, recycled material such as recycled plastic can be used as a tube material. The applicator tip can be made of a different material from the applicator tube. In certain embodiments, the applicator tip can be made of the same material as the applicator tube. The applicator tip can be configured to be attached to the tube via a tube neck or nozzle. In other embodiments, the applicator tip can be integral with or extruded with the tube.

When dispensing a composition from a tube, the tube opening must be dimensioned depending on properties of the composition such as viscosity, surface tension and properties of the spout of the tube. The applicator tip can also be detachable from the neck or nozzle of a tube, such that it may be replaced by a different applicator tip.

The applicator tip can have a surface that is configured to hold an effective amount of composition and allow a subject to apply this amount directly to a facial area in a massaging motion.

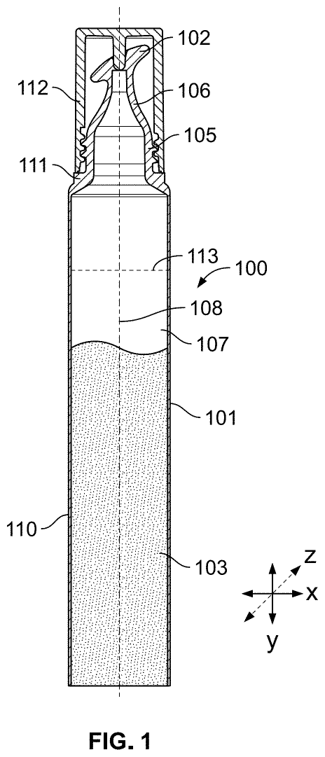

Referring to FIG. 1, a system 100 for modulating facial pain can include a tube 101 having an applicator tip 102 and a composition 103 formulated as a topical balm dispensable from the applicator tip. The device can be used as a tube such as a cosmetic tube. Such tubes have properties that keep product from UV, moisture, and oxygen exposure thereby providing protection of a contained product and increasing product shelf life particularly for sensitive formulations such as those that contain SPF, essential oils, natural cosmetic and skincare formulations. The device can include a nozzle or neck 105 that connects the tube to the applicator tip. The nozzle can have a tapered section 106 that restricts the flow of a contained composition prior to reaching the applicator tip. In some embodiments, a body of a tube can have a length 108 disposed along a y-axis and have a sealed end 109. The device can have a width 113 disposed along an x-axis perpendicular to a y-axis. In some embodiments, a tube's diameter can vary along a z-axis. In certain embodiments, it can remain constant. The device can be made of a first material 110. In some examples, the nozzle can be made of a second material 111. The applicator tip is connected to the tube but structured to allow the composition to be dispensed through an opening and yet remain positioned on a surface surrounding that opening for direct topical application. The applicator tip can be flat or angled with rounded or smooth edges for ease of direct topical application. The applicator tip can be made of a third material. In some examples, each of the first material, second material and third material can be distinct. In some examples, the first material and second material can be distinct. In some examples, the second material and third material can be distinct. In some examples, the first material and third material can be distinct. In some examples, each of the first material, second material and third material can be the same. In some examples, any combination of the first, second or third material can be the same.

To apply the claimed composition, an effective amount or dose can be squeezed or advanced from the device into a nozzle. The effective amount or dose can rest on a surface of the applicator tip, which is configured to hold an effective amount of composition. The subject can then contact the applicator tip to a facial area to apply an effective amount of composition directly to a facial area in a massaging motion, without the need to contact the composition with one's fingers or hands, thereby avoiding unwanted contamination or inadvertent contact with the eye area.

Once applied, effective doses of the compositions of the present subject matter are useful for treating jaw pain, TMJ disorders, underlying muscle pain, or bruxism, which can cause sleeplessness, stress, and severe discomfort.

Using the device and applicator tip, therapeutically effective doses of the compositions of the claimed subject matter provide analgesic effects and promote beneficial health effects including improved skin conditions by providing a composition with antioxidant and anti-inflammatory properties without the need for touching the composition with one's fingers or hands prior to administration.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art.

### EXAMPLES

In order that this invention may be better understood, the following examples are set forth. These examples are for purposes of illustration only and are not to be construed as limiting the scope of the invention in any manner

### Example 1: Preparation of Carrier

In this example, the carrier material includes jojoba oil and beeswax. A piece of beeswax can be melted over low heat (155° F.) and added to jojoba oil. The beeswax was heated in a beaker in a water bath with the water temperature reaching 170-179° F. until the wax is melted. The mixture can be cooled to about 130° F. prior to adding the botanical formulation. Exemplary carriers are shown below. The amount of the carrier can be scaled up or down based on the ratios presented herein.
Carrier Blend A
   4 g beeswax
   4 Tbsp jojoba oil
Carrier Blend B
   1 oz (2 Tbsp) of jojoba oil
   5 g of beeswax
   Carrier Blend C
   1 oz (2 Tbsp) of jojoba oil
   6 g of beeswax

### Example 2: Preparation of Botanical Formulation

After the carrier blend is heated and dissolved, a botanical formulation can be combined in a separate beaker and water bath. The botanical formulation combines the selected oils and crystals, and agitates the mixture at about 125° F. for about 5-7 minutes with the water temperature reaching 170-17° F. After the agitation, the botanical formulation can then be combined with the carrier blend. The two mixtures can be combined to create a composition that can be cooled to room temperature.

Exemplary formulations of the present disclosure are shown below. The formulation can be scaled up or down based on the ratios of the ingredients presented herein.
Formulation A—Botanical Formulation+Carrier
   arnica oil: 0.5 tsp
   wintergreen essential oil: 1 ml (20 drops)
   menthol crystals organic crushed: 0.25 tsp
   camphor crystals: 0.25 tsp
   ginger essential oil: 1 ml (20 drops)
   jojoba oil: 2 Tbsp (1 ounce)
   beeswax: 6 grams
   Total yield product: 21-25 grams
Formulation B—Botanical Formulation+Carrier
   arnica oil: 0.5 tsp
   wintergreen: 0.5 tsp
   menthol crystals crushed: 0.5 tsp
   camphor crystals: 0.25 tsp
   ginger: 0.25 tsp
   lavender: 0.5 tsp
   beeswax: 4 g
   jojoba oil: 33 g
   Total yield product: 42 grams
   The product should have 2.4% menthol and 1.2% camphor which complies with the USP monograph.
Formulation C—Botanical Formulation+Carrier
   arnica oil: 2.13 g
   wintergreen: 2.6 g
   menthol crystals crushed: 1 g
   camphor crystals: 0.63 g
   ginger: 0.25 tsp
   lavender: 1.5 g
   beeswax: 4 g
   jojoba oil: 33 g
   Total yield: 45 g
   The product should have 2.2% menthol and 1.4% camphor and 5.7% wintergreen, which complies with the USP monograph.
Formula D—Botanical Formulation
   arnica oil: 0.5 tsp
   wintergreen: 0.5 tsp
   menthol crystals crushed: 0.5 tsp
   camphor crystals: 0.25 tsp
   ginger: 0.5 tsp
   lavender: 0.75 tsp
Formula E—Wintergreen Free (Salicylate-Free) Botanical Formulation
   arnica oil: 0.5 tsp
   menthol crystals crushed: 1.47 g
   camphor crystals: 0.25 tsp
   ginger: 0.25 tsp
   lavender: 0.5 tsp
   beeswax: 4 g
   jojoba oil: 33 g
   Removal of wintergreen requires a higher menthol content to compensate. The result is a salicylate-free composition.
Formula F—Arnica Free Botanical Formulation
   wintergreen: 18.2 g
   menthol crystals crushed: 7.98 g
   camphor crystals: 4.41 g
   ginger: 7 g
   lavender: 14 g
   beeswax: 27.02 g
   jojoba oil: 231 g
   Total yield: 309.61 g (11 oz)
Formula G—Arnica Free Herbal Formulation
   wintergreen: 14 g
   menthol crystals crushed: 7.98 g
   camphor crystals: 4.41 g
   ginger: 9 g
   lavender: 14 g
   beeswax: 27.02 g
   jojoba oil: 231 g
   Formulation yields 4.5% wintergreen, 2.4% menthol and about 4% lavender and about 2% ginger oil and less than 2% camphor
   Total yield: 305 g (10.759 oz)
Formula H—Botanical Formulation+Carrier
   wintergreen: 18.2 g (5.6%)
   menthol crystals crushed: 7.98 g (2.45%)
   camphor crystals: 4.41 g (1.35%)
   ginger: 9 g (2.15%)
   lavender: 14 g (4.3%)
   beeswax: 27.02 g (12.9%)
   jojoba oil: 162-231 g (50-71.2%)
   Total yield: 324.59 g
Formulation I—Botanical Formulation+Carrier (TJz Balm™)
   menthol crystals crushed: 2.46%
   camphor crystals: 1.36%
   ginger root oil: 2.15%
   lavender: 4.31%
   beeswax: 15.00%
   jojoba oil: 69.12%
   Total yield: 100.00%

### Example 3: FDA Compliance for Botanical Drug Development

Due to the unique nature of botanical drugs, the FDA Center for Drug Evaluation and Research's (CDER) has specific regulations for over the counter drugs. Because of the heterogeneous nature of a botanical drug and possible uncertainty about its active constituents, one of the critical issues for botanical drugs is ensuring that the therapeutic effect for marketed drug product batches is consistent. The pharmacology and toxicology requirements for an NDA for a botanical drug are anticipated to be the same as those for a nonbotanical drug and overall requirements for demonstrating a botanical drug product's efficacy and safety are the same as those for other drug products. Because there could likely be more than one chemical constituent in a botanical drug or the active constituents may not be identified, standard in vivo bioavailability and pharmacokinetic studies that measure the blood or urine concentration of the active moieties or active metabolites may be difficult or impossible to perform. See FDA Botanical Drug Development Guidance for Industry, December 2016.

A botanical drug that has been marketed for a material time and to a material extent for a specific OTC indication may be eligible for consideration in the OTC drug monograph system. To be included in an OTC drug monograph, a botanical drug substance must be recognized in an official United States Pharmacopeia and National Formulary (USP-NF) drug monograph that sets forth its standards of identity, strength, quality, and purity.

The FDA's requirements were complied with for documentation of the methods evaluated and well-controlled clinical studies that establish the botanical drug's safety and efficacy. The tests and specifications were met as regulated by the FDA under an over-the-counter (OTC) monograph for topical analgesics including for menthol as an active ingredient. This includes a botanical raw material control, quality control by chemical tests or process validation, and biological assay and clinical data.

### Example 4: Clinical Efficacy—RIPT Test and Dermatologist Review

A Human Repeat Insult Patch Test or “Repeat Insult Patch Test” (RIPT) was conducted in a single-center, randomized, controlled, single-dose study to determine the sensitization potential of the present composition (Formulation I) on normal skin. The objective of the study was to determine irritation and sensitization potential of the composition as a test material after repeated application to skin. Contact sensitization was measured by treatment emergent adverse events (TEAE5), reaction grade and investigator assessment of irritation and sensitization.

An informed consent was obtained from each volunteer prior to initiating the study. The consent form described reasons for the study, possible adverse effects, associated risks and potential benefits of the treatment and their limits of liability. The following test panel was used:
Number of subjects enrolled: 55
Number of subjects completing study: 51
Age Range: 19-63
Sex:
   Male: 8
   Female: 43
Fitzpatrick Skin Type*
   1—always burn, does not tan: 0
   2—burn easily, tan slightly: 0
   3—burn moderately, tan progressively: 51
   4—burn a little, always tan: 0
   5—rarely burn, tan intensely: 0
   6—never burn, tan very intensely: 0

The present composition was tested under occlusive conditions and placed on an 8-millimeter aluminum Finn Chamber (Epitest Ltd. Oy, Tuusula, Finland) supported on Scanpor Tape (Norgesplaster A/S, Kristiansand, Norway). Alternatively, an 8-mm filter paper coated aluminum Finn Chamber AQUA can be supported on a thin flexible transparent polyurethane rectangular film coated on one side with a medical grade acrylic adhesive, consistent with adhesive used in state-of-the-art hypoallergenic surgical tapes or a 7 mm IQ-ULTRA closed cell system. Such a closed cell system is made of additive-free polyethylene plastic foam with a filter paper incorporated (supplied in units of 10 chambers on a hypoallergenic non-woven adhesive tape having a width of 52 mm and a length of 118 mm) or other equivalents.

The composition (e.g., Formulation I) was tested under semi-occlusive conditions after being placed on a test strip with a Rayon/Polypropylene pad or on a 7.5 mm filter paper disc affixed to a strip of hypoallergenic tape (Johnson & Johnson 1 inch First Aid Cloth Tape). Test materials to be tested in an open patch were applied and rubbed directly onto the back of the subject. Approximately 0.02-0.05 mL (in case of liquids) and/or 0.02-0.05 gm (in case of solids) of the test material was used for the study. Liquid test material was dispensed on a 7.5 mm paper disk, which fit in the Finn Chamber.

The following procedure was applied:
1. Subjects were requested to bathe or wash as usual before arrival at the facility.
2. Patches containing the test material were then affixed directly to the skin of the intrascapular regions of the back, to the right or left of the midline and subjects were dismissed with instructions not to wet or expose the test area to direct sunlight.
3. Patches remained in place for 48 hours after the first application.
4. Subjects were instructed not to remove the patches prior to their 48 hour scheduled visit.
5. Thereafter, subjects were instructed to remove patches 24 hours after application for the remainder of the study.
6. This procedure was repeated until a series of nine (9) consecutive, 24-hour exposures had been made three (3) times a week for three (3) consecutive weeks.
7. Prior to each reapplication, the test sites evaluated by trained laboratory personnel.
8. Following a 10-14 day rest period a retest/challenge dose was applied once to a previously unexposed test site. Test sites were evaluated by trained laboratory personnel 48 and 96 hours after application. In the event of an adverse reaction, the area of erythema and edema were to be measured. Edema is estimated by the evaluation of the skin with respect to the contour of the unaffected normal skin. Subjects were instructed to report any delayed reactions that might occur after the final reading.

Scoring scale and definition of symbols shown below are based on the scoring scheme according to the International Contact Dermatitis Research Group scoring scale:
0: no reaction (negative)
1: erythema throughout at least % of patch area
2: erythema and induration throughout at least % of patch area
3: erythema, induration and vesicles
4: erythema, induration and bullae
D: Site discontinued
Dc: Subject discontinued voluntarily
DcI: Subject discontinued per Investigator

No negative or adverse reactions were reported during the course of this study. There were three (3) subjects with a Grade 4 reaction, one (1) subject with a Grade 3 reaction, four (4) subjects with a Grade 2 reaction, twelve (12) subjects with a Grade 1 reaction and one (1) subject with a delayed Grade 2 reaction to the positive control (2.0% Sodium Lauryl Sulfate Solution). No subjects showed any signs of reaction to the negative control (DI Water).

The study concluded that there was no indication of a potential to elicit dermal irritation or sensitization.

### Example 5: Nighttime Bruxism Studies

The claimed composition (Formulation I) was tested on 9 subjects who suffered nighttime bruxism. The clinical subjects applied the composition for seven consecutive days. The subjects were asked to rate their pain rating on a scale of 0 (no pain)-10 (most severe). Pain levels were measured before and after application. The following results were observed.

| | Pain Level Before | Pain Level After |
|---|---|---|
| Subject | Applying Composition | Applying Composition |
| 1 | 4 | 2 |
| 2 | 10 | 4 |
| 3 | 6 | 2 |
| 4 | 4 | 2 |
| 5 | 8 | 4 |
| 6 | 8 | 0 |
| 7 | 4 | 1 |
| 8 | 4 | 2 |
| 9 | 4-10 | 2 |

The study showed an average pain reduction score of 4 with no adverse reactions.

What is claimed is:

1. A system for relieving jaw pain or bruxism, comprising:
a tube having an applicator tip; and
a composition formulated as a topical balm dispensable from the tube through the applicator tip, the composition comprising a botanical formulation, the botanical formulation comprising an effective amount of menthol, an effective amount of a terpenoid, an effective amount of a lavender oil, an effective amount of a wintergreen oil, and an effective amount of a ginger root oil.

2. The system of claim 1, wherein the composition further comprises a topically acceptable carrier.

3. The system of claim 2, wherein the carrier includes jojoba oil and beeswax.

4. The system of claim 1, wherein the botanical formulation comprises about 1.25 to 16 wt % menthol, 0.1-7 wt % camphor, 2-10 wt % lavender oil, 0-11 wt % wintergreen oil, and 1-8 wt % ginger root oil, based upon the total weight of the composition.

5. The system of claim 1, wherein the carrier comprises 50-74 wt % jojoba oil and 10-20 wt % beeswax, based upon the total weight of the composition.

6. The system claim 1, wherein the composition further comprises one or more of willow bark oil, clove oil, lemon oil, cinnamon bark oil, rosemary oil, and eucalyptus oil.

7. The system of claim 1, wherein the lavender oil is obtained from the leaf, root, flowers, bud or plant of Lavandula angustifolia, Lavandula burnamii, Lavandula dentate, Lavandula dhofarensis, Lavandula latifolia, Lavandula officinalis, Lavandula stoechas, or combinations thereof.

8. The system of claim 1, wherein the terpenoid has an analgesic function.

9. The system of claim 1, wherein the terpenoid is camphor.

10. A method of alleviating jaw pain or bruxism in a human subject, comprising applying to the jaw area of the subject a topical composition comprising a botanical formulation wherein the botanical formulation consists of menthol, a terpenoid, a lavender oil, a wintergreen oil, and a ginger root oil.

11. The method of claim 10, wherein the composition further comprises a topically acceptable carrier.

12. The method of claim 11, wherein the carrier includes jojoba oil and beeswax.

13. The method of claim 10, wherein the terpenoid is an analgesic.

14. The method of claim 13, wherein the terpenoid is camphor.

15. A composition formulated as a topical balm for administration on a human subject, comprising (i) a botanical formulation consisting of menthol, a terpenoid, lavender oil, wintergreen oil, and ginger root oil and (ii) a topically acceptable carrier.

16. The composition of claim 15, wherein the carrier includes jojoba oil and beeswax.

17. The composition of claim 15, wherein the botanical formulation consists of about 1.25 to 16 wt % menthol, 0.1-7 wt % camphor, 2-10 wt % lavender oil, 0-11 wt % wintergreen oil, and 1-8 wt % ginger root oil, based upon the total weight of the composition.

18. The composition of claim 15, wherein the carrier includes 50-74 wt % jojoba oil and 12.9 wt % beeswax.

19. The composition of claim 15, wherein the botanical formulation comprises about 2.45 wt % menthol, 1.35 wt % camphor, 4.3 wt % lavender oil, 5.6 wt % wintergreen oil, and 2.15 wt % ginger root oil.

20. The composition of claim 15, wherein the terpenoid is camphor.
